(19) United States
(12) Patent Application Publication
(10) Pub. No.: US 2010/0280318 A1
(43) Pub. Date: Nov. 4, 2010

(54) Colonoscope Guide and Method of Use for Improved Colonoscopy

(76) Inventor: Stanley F. Chang, Fresno, CA (US)

Correspondence Address: RICHARD A. RYAN;ATTORNEY AT LAW, 8497 N. MILLBROOK AVENUE, SUITE 101, FRESNO, CA, 93720, US

(21) Appl. No.: 12/837,495

(22) Filed: Jul. 15, 2010

Related U.S. Application Data
(63) Continuation-in-part of application No. 11/496,375, filed on Jul. 31, 2006, now Pat. No. 7,798,957.

(51) Int. Cl. A61B 1/00

(52) U.S. Cl. 600/114

(57) ABSTRACT

A guide and method for tilting a colonoscope in the rectum leftward toward the descending colon to advance the colonoscope through the colon during colonoscopy. The guide comprises a rigid elongated body member and a handle attached to or integral with the body member. The body member has an insertion length selected so as to not be inserted past the patient's rectum. The body member can be curved or tubular to better engage the colonoscope. A soft liner can be utilized to protect the endoscope during use. Preferably, the body member has a semi-circular cross-section with a flexible inner tubular member attached thereto that receives the endoscope. A longitudinal slot facilitates loading the endoscope into the tubular member. In another embodiment, the body member comprises upper and lower rigid body members connected by an engaging mechanism. The user tilts the colonoscope with the guide to minimize or avoid sigmoid looping.

### CROSS-REFERENCE TO RELATED APPLICATIONS

This patent application is a continuation-in-part of U.S. patent application Ser. No. 11/496,375 filed Jul. 31, 2006.

### STATEMENT REGARDING FEDERALLY SPONSORED RESEARCH

Not Applicable.

### REFERENCE TO A SEQUENCE LISTING, A TABLE OR A COMPUTER PROGRAM LISTING APPENDIX SUBMITTED ON A COMPACT DISC

Not Applicable.

### BACKGROUND OF THE INVENTION

1. Field of the Invention

The field of the present invention relates generally to devices and procedures for use in endoscopic examinations of the human colon. More specifically, the device and method of the present invention relates to medical equipment and procedures used to overcome the difficulties encountered with looping of the sigmoid during a colonoscopy. Even more specifically, this invention relates to guiding devices and methods of their use which facilitate the insertion of the endoscope into and through the human colon by tilting the endoscope to prevent or overcome problems with sigmoid looping. The method disclosed herein is applicable to both conventional colonoscopy and various modifications thereto, including but not limited to retrograde enteroscopy and automated colonoscopy.

2. Background

As used herein, the term “endoscope” or “scope” is used to refer to a endoscope, colonoscope, sigmoidoscope, proctoscope, gastroscope or other types of medical endoscopes that are or can be utilized to perform a colonoscopy procedure. As known to those skilled in the art, a typical endoscope generally comprises a connecting tube, a handle and an insertion tube (the part inserted into the patient). In the text, the term “scope” generally refers to the insertion tube portion of the endoscope. In referring to the opposite ends of the scope or the guide of the present invention, the “proximal end” refers to that part of the scope or guide which is closest to the operator or physician endoscopist (hereinafter collectively referred to as “operator”) performing the procedure, and the “distal end” refers to that part of the scope or guide farthest from the operator or physician endoscopist. Although medically the rectum is generally not considered to be part of the colon, which includes the sigmoid colon, descending colon, transverse colon and ascending colon, for purposes of this disclosure general references herein to the term “colon” include the rectum. The terms “splint”, “splinting device”, “splinting overtube” and “overtube” are used interchangeably to refer to a generally elongated hollow tubular member that is adaptable for receiving a medical instrument, such as an endoscope, therein to facilitate movement of the scope through the colon. In colonoscopy, these splinting overtubes are all utilized to overcome the problem of looping in the sigmoid colon. These overtubes are all flexible in order to extend from the anus to the descending colon, forming a bridge to prevent the sigmoid from looping, thus facilitating scope advancement.

Colonoscopy is the most sensitive and specific means for examining the colon, particularly for the diagnosis of colon cancers and polyps. Because the cecum, the portion of the colon farthest from the anus, can be a common location for cancer, it is generally desirable that the entire colon be completely examined. During a typical colonoscopy procedure, the scope is inserted into the anus, through the rectum, and then advanced through the sigmoid colon, descending colon, transverse colon, ascending colon and into the cecum. In colonoscopy, straightness of the endoscope is necessary, or at least highly desirable, for the advancement of the endoscope through the colon. Advancing the scope, which is typically about 160 centimeters in length, can be difficult due to a loop or bend in the sigmoid colon. In fact, the advancement of the scope during the colonoscopy procedure often results in the formation of a loop along the shaft of the flexible scope. This looping occurs most commonly in the sigmoid colon and, though usually much less frequently, in the transverse colon. As known to those skilled in the art, failure to substantially straighten the loop in the sigmoid colon prior to continuing to advance the scope can cause enlargement of the loop and result in even more difficulty in advancing the scope. This enlarged looping can result in patient pain and damage, including cardiovascular reactions such as hypotension and bradycardia. Forceful pushing of the scope in the colon when loops are present only increases the pain and is usually ineffective in advancing the scope through the colon. Pain increases the need for sedative and analgesic medications, which can expose the patient to more cardiorespiratory risk.

To address the issue of looping in a colonoscopy, the operator typically utilizes a series of maneuvers, referred to herein as the usual colonoscopic maneuvers, that can be summarized as pushing, pulling, rotating (clockwise or counterclockwise), jiggling, inflation, deflation (suction), external abdominal compression and changing the patient's position, which are done in various combinations and/or sequences as necessary or desirable. The scope is inserted into the rectum and gradually advanced with the usual colonoscopic maneuvers through the sigmoid. In a typical colonoscopy, the sigmoid loop is formed as the scope moves inward. The operator will reduce and then straighten the sigmoid loop by withdrawing the scope to a generally straightened position after he or she reaches the descending colon or the transverse colon. Once the loop is substantially straightened, further advancement of the endoscope can usually be accomplished. Unfortunately, it is not uncommon that upon readvancement of the scope, the sigmoid loop has a tendency to reform. The operator will attempt to prevent or limit reformation of the sigmoid loop with abdominal compression and by changing the position of the patient, thereby allowing advancement of the scope to the cecum. As well known, however, the usual colonoscopic maneuvers are ineffective in a certain number of cases. In fact, subsequent reformation of the sigmoid loop is the most troublesome impediment to the advancement of the distal tip of the scope and accounts for many, if not most, of the failures to reach the cecum, which typically results in three to ten percent of colonoscopies.

As the rectum is a midline, cephalocaudally oriented structure, it tends to “pull” the scope back to a cephalad orientation, which predisposes to sigmoid looping. The variable stiffness colonoscopes, such as the Innoflex™ video colonoscope available from Olympus America, Inc., is somewhat helpful in dealing with the problem of sigmoid looping during colonoscopy. However, looping still often occurs because the scope alone cannot be made rigid enough to avoid the natural, default cephalad direction of advancement through the rectum. The use of a sigmoid splint or overtube has also been found to be highly effective in preventing reformation of the sigmoid loop in order to facilitate advancement of the scope through the colon during a colonoscopy. The prior art sigmoid splinting overtubes are mostly inserted over a straightened scope through the rectum and sigmoid into the descending colon to passively form a bridge between the anus and the descending colon so as to facilitate further scope advancement along a straightened ano-descending colon axis. However, despite all usual colonoscopic maneuvers, the colonoscope usually cannot be perfectly straightened (especially at the recto-sigmoid junction) to allow the introduction of a rigid overtube. In this text, “straighten” includes the concepts of “roughly straighten” or “substantially straighten”. Hence, a rigid splint that extends from anus to descending colon, which is roughly 40 cm, does not exist. Another reason for the non-existence of said splint is that during colonoscopy, the colonoscope outside the anus is almost always left “dangling” with a steep curve, making it somewhat unwieldy to try to load the 40 cm rigid overtube onto the scope. These overtubes are, therefore, all fairly flexible in order to be safely inserted into the descending colon. By forming a bridge linking the anus and descending colon, the scope in the rectum will be tilted to the left. However, because flexibility is a pre-requisite for safe insertion through the sigmoid into the descending colon, none of the prior art overtubes are rigid enough to be able to actively tilt the scope in the rectum. Using these prior art splints, the axis of the scope in the rectum is only passively maintained in the oblique position by being aligned with the descending colon. Actively tilting and maintaining this axis of the scope in the rectum is the objective of the present invention. The method of using a short overtube, sufficiently rigid, to actively tilt a flexible endoscope in the rectum without having to insert this overtube beyond the sigmoid has not been previously disclosed, and there is no such device available in the marketplace, past or present.

As is well known, the prior art splints are not easy to use and can cause complications, such as a perforation, when pushing through bends, especially at the recto-sigmoid junction and the sigmoid-descending junction. In addition, when the need for a sigmoid splint arises, the tip of the scope is usually already in the proximal colon. At this point, the operator may opt to remove the scope entirely in order to load the endoscope inside the sigmoid splint, in an end-to-end fashion, and start the procedure over again. A more convenient way, however, is to be able to load the endoscope into the splint without having to remove the scope from the patient. This is done by using a splint with a longitudinal slot along the length of the splint. The slotted splint is loaded onto the endoscope in a side-to-side fashion by opening the slot, such that the scope does not have to be removed from the patient. Once placed around the scope, the slot is closed. The slot will then need to be fastened or locked in the closed position. A fastening or locking mechanism is needed to keep the splint closed in a secure fashion. There are many patents that teach the use of overtubes/splints for colonoscopy, including U.S. Pat. No. 5,779,624, U.S. Pat. No. 5,941,815 and U.S. Pat. No. 6,712,755, all to Chang (the present inventor).

Yet another disadvantage of the prior art splint is its length of about 40 cm. When it is loaded onto the colonoscope, typically of 160 cm length, much of the scope length cannot be grasped and manipulated by the operator. The guide of the present invention is much shorter and therefore easier to use.

The old-fashioned rigid proctoscope/sigmoidoscope has existed for decades. These rigid scopes are used for the purpose of examining the rectum and part of the sigmoid. When in the rectum, they have to be angled in various directions, often quite severely, to gain optimal views. The fact that these devices have been in use for many years shows that tilting a round, rigid, cylinder-shaped instrument in the rectum is safe. However, these rigid proctoscopes and sigmoidoscopes directly tilt the rectum. They do not tilt flexible endoscopes and have never been designed or designated for facilitating the advancement of a flexible endoscope through the colon. As such, therefore, they do not provide a soft inner surface to allow a flexible endoscope to slide within it without damaging the flexible scope.

A video proctoscope is described in “Practical Gastrointestinal Endoscopy”, by Peter Cotton and Christopher Williams, 4th edition, p. 219. A proctoscope is first inserted into the distal rectum. The insertion trocar is removed. A flexible colonoscope is next inserted into the proctoscope. The colonoscope provides a source of illumination and an excellent close-up view of the distal rectum and anus, especially of hemorrhoids. This combination is used to gain a good view of the distal rectum, but not to facilitate colonoscopy. Specifically, no mention is made in this text of using the proctoscope to tilt the path of the colonoscope. Furthermore, the short proctoscope shown in the illustration (FIG. 9.22) is insufficient in length to tilt the colonoscope at or near the rectum-sigmoid junction in order to facilitate colonoscopy. The rigid anoscope is of course not provided with a soft inner surface to protect the flexible endoscope since the colonoscope is used only to view the hemorrhoids and does not have to slide against the anoscope.

Despite the prevalence of specially configured endoscopes and splints to reduce sigmoid looping in order to improve the effectiveness and reduce the pain of colonoscopy procedures, many operators still face significant difficulty and, on occasion, are unable to reach the cecum due to looping of the sigmoid. As a result, therefore, what is needed is a device and a method of using that device that improves the use of an endoscope during a colonoscopy by reducing the problems associated with insertion and advancement of the scope through the colon. The improved device and method should simplify the colonoscopy procedure and reduce the operator labor and patient discomfort presently associated with colonoscopies that result from sigmoid looping. Preferably, the device should be easy and safe to use and adaptable for use at various stages of the colonoscopy.

In summary, the inventor has determined the following: (1) to make colonoscopy easier, a straightened left-oblique axis from anus to descending colon, as shown by the effectiveness of the prior-art sigmoid splint, should be maintained; (2) because of its flexibility, however, the current colonoscope tends to head cephalad in the rectum when advanced against frictional resistance upstream and that this cephalad vector predisposes to the formation of a sigmoid loop; (3) if the axis of the scope in the rectum can be tilted and kept leftward by using a rigid guide, then the desired oblique axis can be maintained; and (4) by directing the orientation of the scope within the rectum, the same effect as a splinting overtube can be attained without the need to go beyond the rectum or sigmoid.

What is needed is a short overtube or other colonoscope guiding device that is sufficiently rigid to tilt the colonoscope in the rectum. Those skilled in the art will appreciate that the advantages of such an overtube or device include the following: (1) it is not necessary to cross the recto-sigmoid junction or the sigmoid-descending junction, thereby making perforations extremely unlikely; (2) the overtube/device can function even when a mild bend still exists at the recto-sigmoid junction, which is not the case with the prior art splint, such that when the guide tilts the scope in the rectum the angle at the recto-sigmoid junction is diminished, making advancement easier; (3) by working within the rectum alone, the overtube/device can generate a more favorable angle for scope advancement through the sigmoid and descending colons; (4) the rigidity and short length of such an overtube/device make a longitudinal slot more feasible, such that side-loading can be accomplished, which is highly desirable because the scope does not have to be removed from the colon in order to load the device; and (5) the short length takes up much less length of the scope, so that handling of the scope is much easier compared to the prior art longer splints. In addition, during use of the prior art splints the scope has to be first introduced into at least the descending colon, then straightened, before the splint can be introduced. An advantage of the desired overtube/device is that it can also help the initial introduction of the scope through the sigmoid and descending colons. Therefore, the present invention can achieve more than the prior art splint.

### SUMMARY OF THE INVENTION

The colonoscope guide and method of use for improved colonoscopy of the present invention provides the benefits and solves the problems identified above. The colonoscope guide, also interchangeably referred to herein as a “guide” or “vector guide” device, and method of the present invention are based on the anatomical consideration that pushing an unaided flexible endoscope through the rectum moves the scope forward in the craniocaudal axis and on the geometrical consideration that pushing a flexible endoscope in the craniocaudal axis tends to cause looping of the sigmoid. The guide is configured for tilting the scope in a generally oblique direction in the rectum, typically toward the left side of the patient, so as to reduce the difficulty of inserting and advancing the scope in and through the colon. The guide of the present invention simplifies the performance of a colonoscopy by directing the scope into the colon in a manner that reduces sigmoid looping or bending and generally prevents reformation of the loop during the procedure. The method of the present invention moves the direction of scope insertion away from the craniocaudal axis into an oblique axis, to at least approximately align the rectum with the descending colon. In this manner, advancing the scope through the sigmoid will become easier by reducing angulations at the recto-sigmoid junction, the sigmoid and at the sigmoid-descending colon junction. Once the scope tip is in the descending colon or beyond, looping of the sigmoid will also be minimized or eliminated. Various embodiments are disclosed to enable this active tilting of the scope in the rectum toward the descending colon. As such, the guide of the present invention reduces operator labor to perform the colonoscopy and reduces the pain and discomfort of the patient that would otherwise result from sigmoid looping. In its preferred embodiment, the guide of the present invention is relatively inexpensive to manufacture and adaptable for use when the scope is inserted into the anus and/or during later advancement of the scope through the colon. The method of the present invention, utilizing the above device, is relatively simple to follow and effective at improving the likelihood of reaching the cecum while reducing patient discomfort and pain.

The method of the present invention actively tilts and/or maintains the tilt of the scope from within the rectum without having to link the anus with the descending colon, as in the prior art. In order to do so, at least the proximal segment of the device of the present invention has to be rigid, and this segment has to be continuous with or connected to the handle, which is used to tilt the device. The rigid component of the device of the present invention has an insertion length of no greater than 25 cm, functions in the rectum alone or, at most, also in the distal sigmoid. The rigid section, which does the work of tilting the scope, will not reach the descending colon. Thus, this mechanism is distinctly different from the existing prior art sigmoid splinting overtubes.

As a result, the requirements for the guide of the present invention include the following: (1) the guide has an elongated body member, preferably an overtube, in order to eliminate the antero-posterior curvature of the rectum against the sacrum; (2) at least a sufficient portion of the circumference of the guide (e.g., 10%) has to be rigid, which is defined as being sufficiently rigid to tilt the scope; (3) the rigid portion of the guide should be approximately 5 to 25 cm in length; (4) the guide has a handle to effect the tilting and maintenance of the tilt; (5) the handle has to be continuous, whether integral or attached, with the rigid portion of the guide; (6) the handle also serves the purpose of preventing complete entry of the guide into the rectum; (7) as the scope will be sliding and rubbing against the inner surface of the guide, the inner surface needs to be soft or otherwise configured so as to not damage the scope; (8) the soft inner layer of the guide does not have to be the same length as the rigid layer (for instance, it is generally preferred that the soft layer be longer in order to protect the scope from rubbing against the edges of the rigid layer; (9) the inner surface needs to be lubricious and/or “Bumps” can be built onto the inner surface to reduce surface area and to help retain the lubricant applied onto the scope by the operator; (10) the guide can include a longitudinal slot in order to enable side-loading of the scope; and (11) if a slot is utilized, a closure mechanism can be built into the device to allow closure, such as factory-installed adhesive tape, or a sliding rail mechanism.

In one embodiment of the present invention, the colonoscope guide comprises an elongated body member that can be configured to be generally flat, curved or semi-circular having an inner side that abuts against the colonoscope inside the rectum and sigmoid colon. The curved and semi-circular cross-section configurations provide improved engagement with the scope. Further improved engagement is achieved with a wider distal end, a more semicircular shape cross-section and/or the use of one or more ring members or a tubular member to receive the scope and hold the guide in generally abutting relation therewith. A handle is provided at the proximal end of the body member to allow the operator to manipulate the guide so as to direct and maintain the angle of the scope to improve the ability to advance it through the colon by reducing the sigmoid loop and preventing its reformation. The guide can be provided with an injection port hydraulically connected to one or more channels having one or more discharge openings to allow the operator to inject lubricant along the inner surface to improve the guide sliding along the scope or the scope sliding along the guide.

In one preferred embodiment, the body member has an outer layer comprised of a stainless steel or a hard, rigid plastic material, which is elongated, 15 cm in length, and has a cross-section of a half circle. The rigid layer can be bonded to the outer wall of an inner layer, which can be in the form of an overtube having a circular cross-section, that is made out of a softer material in order to avoid damaging the colonoscope. The softer inner layer can be of a different length than the rigid body member, such as being longer to protrude beyond the distal end of the body member. The distal end can be tapered to aid insertion into the rectum over the colonoscope. The colonoscope guide can have a longitudinal slot to allow loading of the guide after the colonoscope has already been inserted into the rectum. In another embodiment, the soft inner layer can be much longer than the rigid body member (e.g., 40 cm versus 15 cm). The rigid layer, not present in the prior art overtubes, is constructed into the proximal segment of the present overtube. The rigid body member segment is continuous with the handle that is used for tilting the colonoscope guide.

To perform a colonoscopy procedure using an endoscope and the guide configured according to an embodiment of the present invention, the operator first preloads the guide, introduces the scope into the colon and then advances it using usual colonoscopic maneuvers. The guide of the present invention can be used to help the scope advance through the sigmoid, and then later to further advance to the cecum or terminal ilieum. By tilting or keeping the scope in the left-oblique direction, the sometimes steep angulations of the recto-sigmoid junction, the sigmoid colon and the sigmoid-descending junction are softened and insertion becomes easier. Once the operator is no longer able to advance the scope or advance it without causing pain or looping, he or she pulls back on the scope until the sigmoid and rectum are essentially straightened and then inserts the guide into the rectum over and along the scope. Using the anus as a fulcrum point, the operator tilts the guide to the patient's left side to enable pushing the scope in that direction so as to better align the rectum and sigmoid colon with the descending colon. With the scope tilted to substantially straighten the left colon, and with this tilt maintained by the guide, the operator then continues with the colonoscopy by advancing the scope through the colon. The colonoscope and the guide are periodically advanced and withdrawn, with the guide tilted from time to time, as needed to maintain a left axis, in order to avert or minimize sigmoid loop formation. In an alternative method of use, the guide, most likely in the form of an overtube or partial overtube, can be first introduced into the rectum. The scope is next inserted or engaged with the guide. The guide and scope are advanced individually or together, while tilting of the guide and manipulation of the scope and guide are carried out. The guide is periodically tilted toward the descending colon so that the scope is maintained in that axis while advancing.

When a guide with a slot is used, the colonoscope is first introduced and advanced using the usual colonoscopic maneuvers. When sigmoid looping becomes problematic, the scope is withdrawn to reduce any sigmoid loops. The guide is next loaded onto the shaft of the scope through the slot. The slot is closed by one of several mechanisms. During its use, lubrication is applied and the guide is slid over the scope through the anus into the rectum. Tilting to the left is carried out as described above to align the anus with the descending colon, and the procedure is completed with the usual colonoscopic maneuvers, along with periodic lubrications, tilting, advancing, and withdrawal of the scope and the guide, as described above.

In summary, the method of the present invention is to utilize a device to actively tilt and/or maintain the tilt of a flexible endoscope in the rectum toward the descending colon to help minimize the formation or reformation of a sigmoid bend/loop in the performance of a colonoscopy. This method has not been previously disclosed.

Accordingly, the primary objective of the present invention is to provide a colonoscope guide and method of use for improved colonoscopy that provides the advantages and features discussed above and which overcomes the disadvantages and limitations associated with presently available devices for and methods of performing colonoscopy procedures.

It is also an important objective of the present invention to provide a colonoscope guide and method of its use that reduces the difficulties associated with performing a colonoscopy which are due to sigmoid looping.

It is also an important objective of the present invention to provide a colonoscope guide and method of its use that simplifies insertion and advancement of an endoscope through a colon having sigmoid looping that reduces operator labor and time to perform the colonoscopy and which reduces patient discomfort and pain associated with such procedures.

It is also an important objective of the present invention to provide a colonoscope guide that is configured to effectively and safely tilt or angle the colonoscope and maintain it in that direction so as to ease its insertion into and advancement through the colon, particularly in light of sigmoid looping that is present in the colon.

It is also an important objective of the present invention to provide a method of performing a colonoscopy that utilizes a colonoscope guide to substantially reduce the effect of sigmoid looping in order to ease the insertion and advancement of the endoscope into and through the colon.

It is also an important objective of the present invention to provide a colonoscope guide having a generally elongated body, with a handle at its proximal end, that is adapted to tilt or angle an endoscope as it enters and advances through the colon during a colonoscopy.

The above and other objectives of the present invention are explained in greater detail by reference to the attached figures and description of the preferred embodiment which follows. As set forth herein, the present invention resides in the novel features of form, construction, mode of operation and combination of parts presently described and understood by the claims.

### BRIEF DESCRIPTION OF THE DRAWINGS

In the drawings which illustrate the best modes presently contemplated for carrying out the present invention:

FIG. 1 is a schematic diagram representing the geometry of a section of the colon, with the segment AB representing the rectum in its natural craniocaudal axis, segment BC representing the sigmoid, segment CD representing the descending colon and segments AE and EC representing the axis change resulting from use of the guide of the present invention;

FIG. 2 illustrates a scope advancing into the sigmoid before use of the guide of the present invention;

FIG. 3 illustrates the scope from FIG. 2 reaching the proximal sigmoid after pushing, pulling, rotating and etc. with the usual colonoscopic maneuvers in the sigmoid to pleat and shorten it, as shown by the accordioned contour of the sigmoid;

FIG. 4 is a prior art configuration illustrating the scope from FIG. 3 forming a loop in the sigmoid because the force of pushing is directed generally cephalad or nearly cephalad;

FIG. 5 illustrates the use of the guide of the present invention to tilt the axis of FIG. 3 to the left side of the patient's body to align the rectum toward the descending colon to allow the operator to advance the scope more easily through the colon while avoiding formation of the sigmoid loop;

FIG. 6 illustrates the scope in the proximal descending colon, with the scope pulled back to generally straighten it (a gentle “S” shape remains);

FIG. 7 illustrates the prior art pushing of the scope from FIG. 6 with a loop resulting because the force of pushing is directed cephalad or nearly cephalad;

FIG. 8 illustrates the guide of the present invention being used with FIG. 6 to tilt the scope so as to generally line up with the descending colon, the axis from the anus to the descending colon being relatively straight and being maintained by the guide of the present invention pushing against the scope as it is efficiently advanced;

FIG. 9 is a side view illustrating one of the preferred embodiments of the colonoscope guide of the present invention showing the body member being wider and having a curved cross-section at the distal end;

FIG. 10 is a side view illustrating an alternative embodiment of the colonoscope guide of the present invention showing use of a ring member near its distal end;

FIG. 11 is a side view illustrating an alternative embodiment of the colonoscope guide of the present invention showing use of a slotted ring member near its distal end;

FIG. 12 is a perspective view of the distal end of a colonoscope guide having a generally flat cross-section and provided with a liner material on its inner surface.

FIG. 13 is a perspective view of the distal end of a colonoscope guide having a generally crescent shaped cross-section;

FIG. 14 is a perspective view of the distal end of a colonoscope guide having a generally semi-circular cross-section;

FIG. 15 is a side view illustrating an alternative embodiment of the colonoscope guide of the present invention showing use of an injection port at the proximal end of the body member hydraulically connected to channels having discharge ports on the inner side of the body member;

FIG. 16 is a side vide of the embodiment of FIG. 14 with the injection port on the handle;

FIG. 17 is a side perspective view of a guide configured according to an alternative embodiment of the present invention showing use of a tubular member at the distal end of the body member;

FIG. 18 is a side perspective view of a guide configured according to an alternative embodiment of the present invention showing use of a slotted tubular member at the distal end of the body member;

FIG. 19 is a side perspective view of a guide configured according to an alternative embodiment of the present invention showing use of a tubular member that extends the entire length of the body member;

FIG. 20 illustrates use of the guide configured according to a tubular embodiment of the present invention being utilized in a natural orifice transluminal endoscopic surgery procedure to perform a splenectomy;

FIG. 21 is an exploded perspective view of a guide configured according to an alternative embodiment of the present invention showing use of a guide having a rigid upper layer, soft intermediate layer, a rigid lower layer and a tube engaging mechanism to join the upper and lower layers together so the guide can direct the colonoscope;

FIG. 22 is an end view of the distal end of the guide of FIG. 21;

FIG. 23 is a side view of a tubular body member, consisting of an outer layer of rigid material, encompassing approximately 50% of the circumference, that is continuous with a handle, and a soft inner layer, which forms a complete tube to house an endoscope;

FIG. 24 is a side view of a tubular body member with a longitudinal slot for side-loading of an endoscope; and

FIG. 25 is a cross-section of a tubular body member showing a rigid outer layer, encompassing approximately 50% of the circumference, and a fully circular soft inner layer having a slot and a plurality of small protrusions on the inner surface to reduce friction and allow retention of some of the lubricant applied on the shaft of the endoscope as the endoscope slides inside the tubular member.

### DETAILED DESCRIPTION OF THE PREFERRED EMBODIMENTS

With reference to the figures where like elements have been given like numerical designations to facilitate the reader's understanding of the present invention, the preferred embodiments of the present invention are set forth below. The enclosed figures and drawings are merely illustrative of a preferred embodiment and represent one of several different ways of configuring the present invention. Although specific components, materials, configurations and uses are illustrated, it should be understood that a number of variations to the components and to the configuration of those components described herein and in the accompanying figures can be made without changing the scope and function of the invention set forth herein.

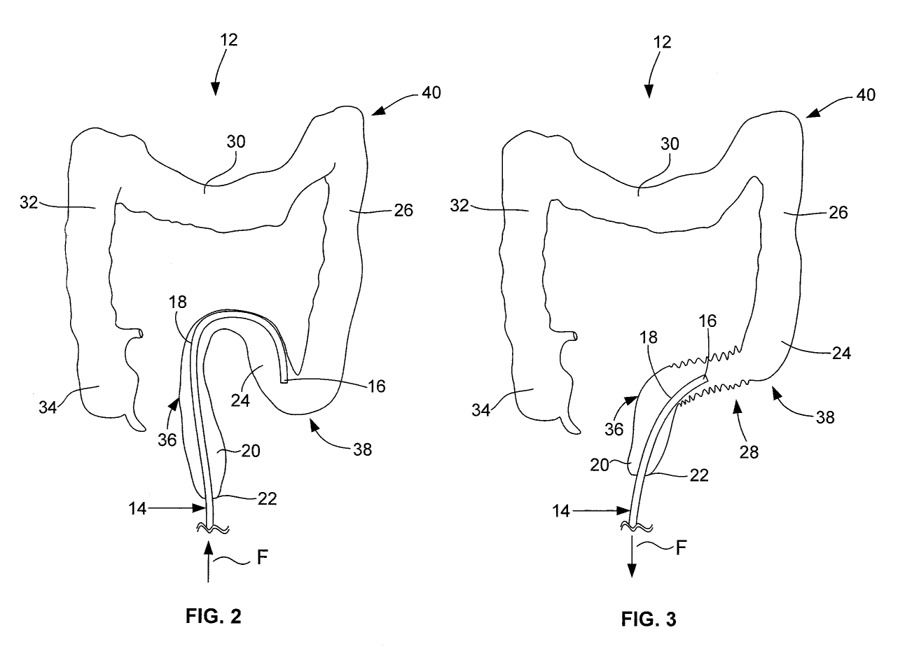

A colonoscope guide that is manufactured out of the components and configured pursuant to a preferred embodiment of the present invention is shown generally as 10 in the figures. As set forth in more detail below, guide 10 is utilized to assist in the investigation of the colon 12, shown in its entirety in FIGS. 2 and 3, with a colonoscope 14 or equivalent medical device (endoscope). The distal end or tip 16 and insertion tube or body 18 of scope 14 are introduced into the rectum 20 through the anus 22 and then into the sigmoid colon 24 (with the arrows “F” on the figures showing the movement of the scope 14 into or out of the colon 12). After negotiating the loop in the sigmoid colon 24, the scope 14 enters the descending colon 26. Further advancement of the tip 16 through the colon 12 is made easier by partially withdrawing the scope 14 to straighten the sigmoid colon 24 with the insertion tube 18 of scope 14, causing the sigmoid colon 24 to take on the accordioned contour 28 (as shown in FIG. 3). Once the sigmoid colon 24 is generally straightened, the tip 16 of the scope 14 is advanced to the descending colon 26, through the transverse colon 30 and ascending colon 32 and then into the cecum 34. After withdrawal of scope 14 and completing the colonoscopy procedure, the scope 14 is removed from the patient.

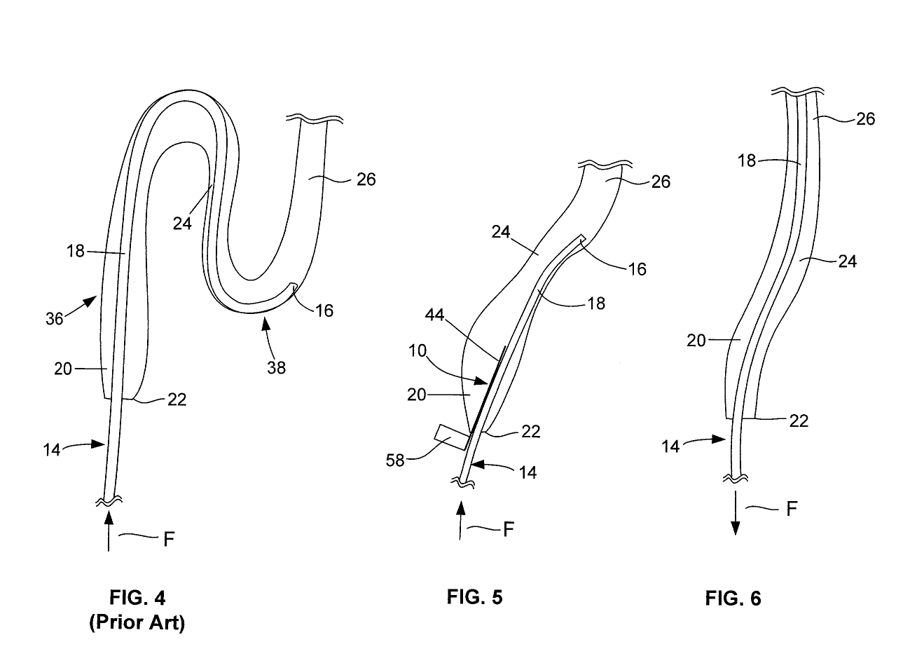

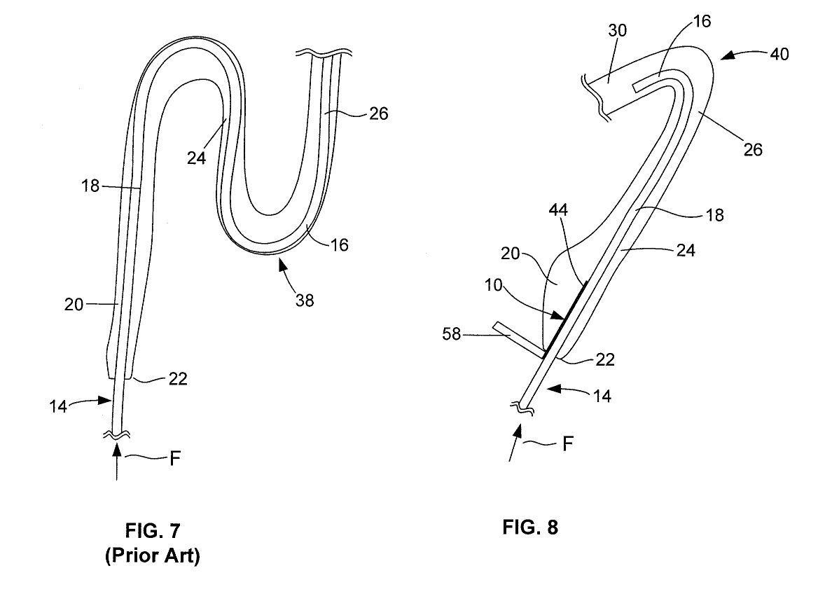

The typical problems that arise in colonoscopy are best shown in FIGS. 4 and 7. At one or more points (shown in FIGS. 2 and 3) during scope 14 advancement through the sigmoid 24, the lumen of the proximal sigmoid 24 and/or the descending colon 26 is clearly and invitingly in view in front of the scope tip 16. However, typically, pushing scope 14 in the prior art fashion (i.e., in the cephalad direction) will not directly move the tip 16 into this lumen. Instead, a loop forms in the sigmoid 24, shown in FIG. 4, before the tip 16 moves forward. This scenario is extremely familiar to all colonoscopists (i.e., the lumen ahead is “so close, yet so far”). Later in the colonoscopy procedure, with tip 16 of scope 14 further upstream and after pulling back to straighten (i.e., as shown in FIG. 6), the prior art cephalad pushing of the scope 14 causes the sigmoid 24 to loop again (shown in FIG. 7). Typically, in the prior art, pushing the scope 14 causes the tip 16 to not move or even move backwards. This is an extremely common scenario in colonoscopy. The reformation of the sigmoid loop can occur repeatedly en route to the cecum 34. The movement of the scope 14 from FIG. 3 to the loop of FIG. 4 and from FIG. 6 to the loop of FIG. 7 is where the patient incurs pain. As explained more in detail below, use of guide 10 of the present invention eliminates or substantially eliminates the loops of FIGS. 4 and 7, such that the colonoscopy proceeds from that shown in FIG. 3 to the condition shown in FIG. 5, skipping the loop of FIG. 4, and from that shown in FIG. 6 to the condition shown in FIG. 8, skipping the loop of FIG. 7.

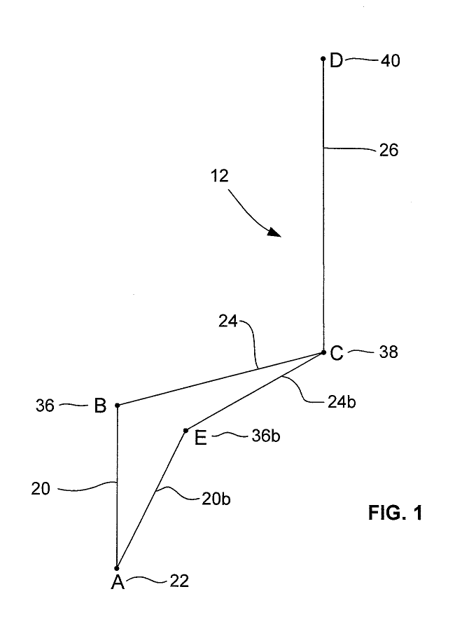

The guide 10 and method of the present invention offers a solution for the problem of sigmoid looping, the looping best shown in FIGS. 4 and 7, in colonoscopy. The cause of sigmoid looping, from the anatomical and geometrical perspective, is that the force of pushing the scope 14 during a typical or prior art colonoscopy is generally limited to the craniocaudal axis (references to the craniocaudal herein are intended to refer to both craniocaudal and nearly craniocaudal). The cephalad direction of push predisposes the colon 12 to sigmoid looping, while the desirable direction of the force is obliquely toward the descending colon 26. The solution presented by the guide 10 and method of the present invention is to tilt and maintain the scope 14 in the rectum 20 more leftward, referencing the left side of the patient's body from the patient's perspective, to allow the force of pushing the scope 14 to be generally directed toward the descending colon 24. This objective is shown in the schematic of FIG. 1, with point A representing the anus 22, line segment AB representing the rectum 20, line segment BC representing the sigmoid colon 24, point B representing the recto-sigmoid junction 36, line segment CD representing the descending colon 26, point C representing the sigmoid-descending junction 38 and point D representing the splenic flexure 40 during a typical or prior art colonoscopy. When a colonoscopy is performed with the guide 10 of the present invention, point E in FIG. 1 illustrates the positioning of the recto-sigmoid junction 36b, or vicinity, at the left wall of the recto-sigmoid junction after the guide 10 improves the alignment of the rectum and sigmoid colon, shown as 20b and 24b respectively after being acted upon by guide 10, with the descending colon 26 to improve the colonoscopy procedure (as set forth in more detail below).

As well known to those skilled in the art, the rectum 20 is a hollow tubular organ situated at about midline in the craniocaudal orientation. Viewed laterally, it is posterior in position and curves anteriorly along the sacrum as it meets the sigmoid 24. It is mostly extraperitoneal, attached by ligaments and fascia, to maintain its craniocaudad orientation. After reaching the descending or transverse colon, by pulling and straightening (which includes the concept of “nearly straightening”) scope 14, the scope 14 in the rectum 20 can be tilted laterally to generally follow the axis of the descending colon 26, but pushing on the insertion tube 18 of scope 14 tends to move the axis of scope 14 in the rectum 20 back to the craniocaudal orientation (whether just one wall of the rectum 20 is tilted by tilting scope 14 or the rectum 20 as a whole is tilted is inconsequential for purposes of this invention). At about 15 cm above the anus 22, the rectum 20 enters the peritoneal cavity, and then shortly thereafter, it meets the sigmoid 24. It is generally irrelevant, for practical purposes, to regard this intraperitoneal segment of the rectum 20 as the rectum 20 or sigmoid 24, but for purposes of this disclosure, the point where the rectum 20 enters the peritoneum is regarded as the recto-sigmoid junction 36, because the rectum 20 becomes a much less mobile organ below that point. The sigmoid colon 24 is extremely variable in length and shape and it connects the top of the rectum 20 to the lower end of the descending colon 26. From the top of the rectum 20, the sigmoid colon 24 heads leftward (or initially rightward and then leftward), anteriorly and then posteriorly, and generally upward, to join the descending colon 26. The sigmoid-descending junction 38 is highly variable in location. The descending colon 26 is mostly retroperitoneal situated longitudinally and has variable lengths of mesentery and thus mobility. The descending colon 26 is generally oriented craniocaudad, roughly parallel to the rectum 20, and separated from the rectum 20 by about half of the width of the abdominal cavity. The recto-sigmoid junction is usually positioned below the sigmoid-descending junction 38, as best shown in FIGS. 2 and 3.

It is generally well known that the rectum 20 can be safely tilted to one side or another by a stiff or rigid instrument, such as the proctoscope or sigmoidoscope, which is often tilted quite severely in order to obtain a good view of the interior of the rectum 20. During the typical prior art colonoscopy, the rectum 20 is tilted to the left when the scope 14 is pulled back and straightened after reaching the descending colon 26 in order to generally align the rectum 20 with the descending colon 26. A sigmoid splint or overtube, when used, keeps the axis from the anus 22 to the mid-to-proximal descending colon 26 a generally straight line in order to avoid sigmoid loop reformation. Unlike the rigid proctoscope/sigmoidoscope, however, a flexible scope 14, which is necessary to achieve advancement through the descending 26, transverse 30 and ascending 32 colons to reach the cecum 34, is not able to effectively tilt the rectum 20. As a result, the pushing force imparted onto a scope 14, shown by the arrows on FIGS. 2 and 4, is directed generally cephalad and usually cannot go obliquely toward the descending colon 26 on its own.

The cause of sigmoid looping can be simply but adequately explained on the basis of geometry and, although colonic anatomy is three-dimensional, the concept can be illustrated in the two-dimensional schematic of FIG. 1. When a scope 14 is introduced into the rectum 20, it first heads cephalad for about 15 cm in the rectum 20, before entering the sigmoid colon 24. The first 10 cm (approximately) of entry into the sigmoid 24 is accomplished by bending the front four-way bending section of the scope 14, as shown in FIG. 2. However, the direction of movement of the insertion tube 18 is generally limited to the craniocaudal axis, as dictated by the anus 22 and rectum 20, causing a loop to be formed, as best shown in FIGS. 2 and 4. The size and complexity of the loop in the sigmoid 24 can be lessened by skillful application of the usual colonoscopic maneuvers discussed above, especially with pulling back, rotation and etc., resulting in pleating (shortening) of the sigmoid 24 into the accordioned contour 28 shown in FIG. 3. The configuration of FIG. 3 is usually obtainable even with long, tortuous sigmoids. Subsequent advancement of the scope 14 into the descending colon 26, again by pushing cephalad as dictated by the anus 22 and rectum 20, often reforms the sigmoid loop, as shown in FIG. 4. Looping on initial insertion through the sigmoid 24 can thus occur more than once during the colonoscopy.

Geometrically, the looping of the sigmoid 24 happens because the craniocaudal axis has practically no lateral component. Thus, lateral movement to get through the sigmoid 24, by pushing craniocaudal, depends on deflecting against a loop in the sigmoid 24. The expertise possessed by many operators, especially by using frequent pull-backs and rotations, enables them to reach the descending colon 26 quickly. However, most operators cannot escape the limitations dictated by the unfavorable geometry of the distal colon 12. Conversely, if the scope 14 can be tilted in the rectum 20 to generate an oblique vector, a lateral component is generated. The tilt itself will “nudge” the scope 14 slightly forward laterally. In addition, the force of further pushing along this oblique axis will also go partly laterally, and will also approach the wall of the sigmoid 24 with a more favorable angle, as shown by FIG. 5. In so doing, passage through the sigmoid 24 is accomplished, partly with the direct force of pushing and partly with wall deflection, against a much less tense wall. Sigmoid looping is thus minimized or avoided.

The guide 10 of the present invention provides a tool for tilting the scope 14 in an oblique direction in the rectum 20, generally toward the left side of the patient. As explained in more detail below, the guide 10 applies external force to the scope 14 inside the rectum 10 to better direct the scope 14 into the descending colon 26 and maintain the scope 14 in that tilted axis. In a preferred embodiment, guide 10 comprises an elongated body member 44, which is sized and configured to be received in the rectum 20 through anus 22.

The prior art sigmoid splinting overtubes are designed to bridge the anus 22, rectum 20, sigmoid 24 and the descending colon 26. Those overtubes are useful only when the scope 14 has first been introduced into at least the descending colon 26. These overtubes and prior art methods of using those overtubes are geared toward solving the problem of sigmoid looping when the scope 14 has already worked its way substantially above the sigmoid 24. They do not address the problem of advancing through a difficult sigmoid 24, which is the other most frequently encountered difficulty in colonoscopy.

Advancing through a tortuous, angulated, fixated, spastic sigmoid 24 is often very challenging. This kind of difficult colon anatomy arises commonly, especially in women with a history of hysterectomy or other pelvic surgeries. These sigmoids 24 are often affected by surrounding pelvic adhesions from previous childbirths, inflammations, and surgical interventions. Another common cause of difficulty in traversing the sigmoid 24 is encountered in severe sigmoid diverticulosis, seen in older men and women.

Just like the looping problem commonly encountered when the scope 14 is up in the proximal portion of the colon 12, this difficulty of getting through the sigmoid 24 and descending colon 26 also occurs because of bending or looping of the sigmoid 24. Similarly, if this bending or looping of the sigmoid 24 can be minimized, then this passage also becomes easier. A very difficult spot to advance through is often the sigmoid-descending junction 38, where the lumen is often outside the view of the lens at the tip of the scope 14, even with maximal deflection of the steerable tip 16. This occurs because this turn is extremely steep (i.e., a hair-pin turn) and advancing the scope 14 through this extremely steep, hairpin turn is challenging. Unfortunately, these tough sigmoids 14, with these severe angulations, occur somewhat frequently in colonoscopy.

The cause of these steep turns is almost always looping of the sigmoid 24. As illustrated in FIG. 4, if the U-turn at the sigmoid loop (the first U-turn) does not occur, then the hair-pin turn at the sigmoid-descending junction 38 (the second U-turn) will not occur. In other words, the first U-turn causes the second U-turn. No first U-turn, no second U-turn.

The present invention addresses this issue. If the scope 14 is inserted through the sigmoid 24 along the desired leftward axis through the rectum 20, then the sigmoid U-turn is avoided. The technique requires use of the tilt or vector guide 10 that is the subject of the present invention. The guide 10 is utilized to direct the axis of the scope insertion in the rectum 20 toward the left side aiming at the descending colon 26. Along with repeated small advances, pull-backs, and scope rotations, the scope 14 no longer needs to form a U-turn to get through the sigmoid 24. This in turn obviates a second U-turn. FIG. 2, shows the scope 14, having traversed the rectum 20, having moved through part of the sigmoid 24. After a series of advances and pull-backs, the scope 14 and sigmoid 24 are substantially straightened, as shown in FIG. 3. If the scope 14 is pushed forward at this point along the natural, cephalad path dictated by the rectum 20, chances are it would form the configuration of FIG. 4, resulting in a double U-turn. If, however, the guide 10 of the present invention is utilized to orient the scope 14 leftward toward the descending colon 26 while the scope 14 is pushed forward from the position of the scope 14 in FIG. 3, then the double U-turn configuration of FIG. 4 is avoided. Instead, the scope 14 will now move much more easily through the sigmoid-descending junction 38 into the descending colon 26, as shown in FIG. 5. Similarly, for further advancement of the scope 14, when done without use of the guide 10 of the present invention, a sigmoid loop as shown in FIG. 7 would result, and advancement of the scope 14 becomes difficult. On the other hand, if the movement is kept leftward from FIG. 5, (i.e. kept along a substantially straight axis from rectum 20 to descending colon 26) with use of guide 10 of the present invention, then further pushing of the scope 14 would result in the configuration of FIG. 8, where the scope 14 is much more easily advanced beyond the descending colon 26 into the transverse colon 30. While this method of insertion still may not generate or require a perfect straight line through the sigmoid 24, it can blunt the sigmoid turn (otherwise a U-turn, or the first U-turn) and, in doing so, generate a much gentler angle at the sigmoid-descending junction 38 for intubation. Again, no first U-turn, no second U-turn, because the angle of the sigmoid turn is commensurate with the angle of the sigmoid-descending junction 38. This move is illustrated in FIG. 5. Water infusion also helps by minimizing the expansion of the sigmoid 24, making it shorter.

In the situation where there is severe diverticular disease of the sigmoid 24, advancement of the scope 14 into the descending colon 26 can be difficult, usually because of a spastic or narrowed lumen, which requires a bit of force for the tip 16 of the scope 14 to push through. When pushing against a narrowed lumen, the tip 16 meets considerable resistance, such that the usual cephalad pushing force, instead of advancing the tip 16, causes formation of a sigmoid loop behind the tip 16 of the scope 14. The sigmoid loop in turn causes additional pushing force to not be directly transmitted to the tip 16. It thus acts in a vicious cycle. Spasm of the colon lumen can be overcome with an injection of Glucagon, but that is sometimes not sufficient. The solution is, again, the same method of using the guide 10 to tilt the scope 14 in the rectum 20 leftward, such that pushing and pulling and scope rotation are all performed along this desirable axis, i.e., aiming at the descending colon 26.

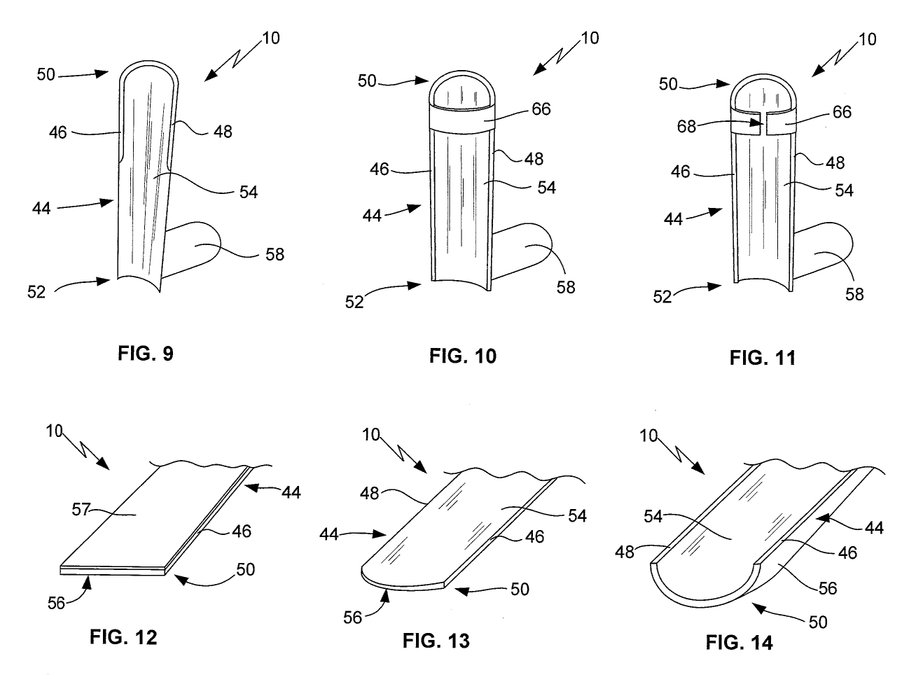

The body member 44 of guide 10 can be straight or slightly curved. If curved, body member 44 preferably has a gentle concavity facing the scope 14 during use. A gentle curve can also be provided to accommodate the curvature of the scope 14 in the rectum 20 along the curvature of the sacrum. In one embodiment, shown in FIG. 12, body member 44 is generally flat or planar in cross-section, having first edge 46 and second edge 48 and relatively thin. In a preferred embodiment, however, at least a portion of body member 44 towards its distal end 50 has a non-planar or curved cross-section with non-planar edges 46 and 48, such as the generally crescent shape shown in FIG. 13 or the half-circle shape shown in FIG. 14, to better maintain contact with the scope 14 when inside the colon 12. In addition, the portion of body member 44 towards its distal end 50 can be made generally wider (i.e., from first edge 46 to second edge 48) in cross-section than the portion of body member 44 at its proximal end 52, as shown in FIG. 9, to further assist with partial enclosure of the scope 14. As set forth in more detail below, to facilitate use of guide 10, body member 44 should be made out of generally stiff material that provides a semi-rigid to rigid guide body member 44 to allow the operator to apply a directing force against scope 14, as described herein, and yet still be able to safely and effectively insert the body member 44 into the colon 12 as far as desired. Body member 44 can be made out of metal, hard plastic or like materials.

The inner side 54 of body member 44, which is the side that contacts scope 14 during use and is opposite the outer side 56, is made or provided with a relatively soft interface, for instance being lined with liner 57 made of a generally softer material such as rubber, silicone or the like, to prevent scraping against scope 14 during use of guide 10 (e.g., when body member 44 is made out of metal). In addition, the inner side 54, or the liner 57 lining it, can be coated with a lubricant. The preferred lubricant is a hydrophilic substance, such as that which is used to line glide-type guide wires. Hydrophilic substances have the unique property of becoming extremely slippery when wet with water or saline. An adequate layer of the hydrophilic substance should be coated and/or impregnated onto the surface of inner side 54 of body member 44 or the liner 57 lining body member 44. Water or saline is applied to this surface to make the sliding surface extremely slippery, thereby allowing easy passage of the guide 10 along scope 14 or the scope 14 along guide 10.

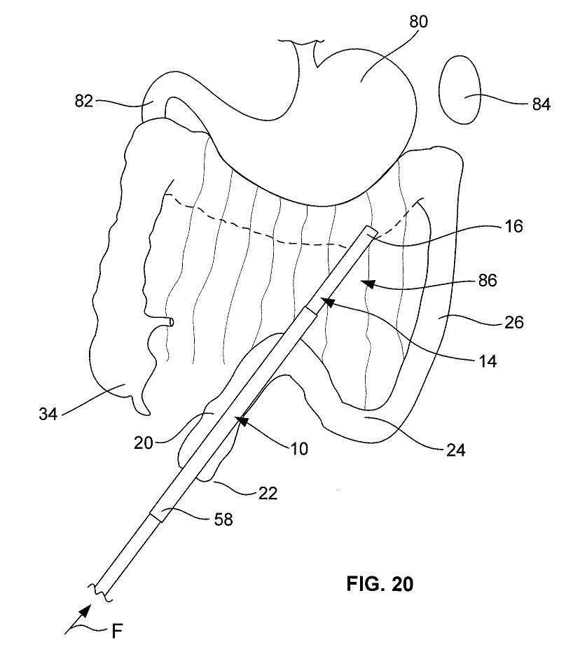

To facilitate tilting guide 10 inside the rectum 20, a handle 58 is provided at or near the proximal end 52 of body member 44, as shown in FIGS. 9-11 and 15-19. The handle 58 can be attached to or integral with the rigid component of body member 44. Preferably, handle 58 is oriented at an angle to the longitudinal axis of body member 44 and it is sized and configured to prevent full entry of guide 10 into the rectum 20, such as having a larger cross-section (i.e., diameter) than body member 44. Handle 58 should be sized and configured so as to be suitable for grasping by the operator's hand, preferably comfortably, to allow him or her to insert, withdraw, rotate, tilt, and un-tilt body member 44. As is readily apparent from the discussion below, movement of handle 58 controls the direction and degree of tilting of guide 10. When tilting is performed during use of guide 10, the handle 58 tilts the elongated body member 44 of the guide 10 by using the anus 22 as the fulcrum. The elongated body member 44 then tilts or pushes the scope 10 into better alignment with the descending colon 26 so as to reduce looping in sigmoid 24. Preferably, handle 58 is oriented at a suitable angle to the body member 44, including being in continuity with it (as shown in FIG. 20), so the operator can know which direction he or she is pushing the scope 14 while body member 44 is inside the colon 12 during the colonoscopy.

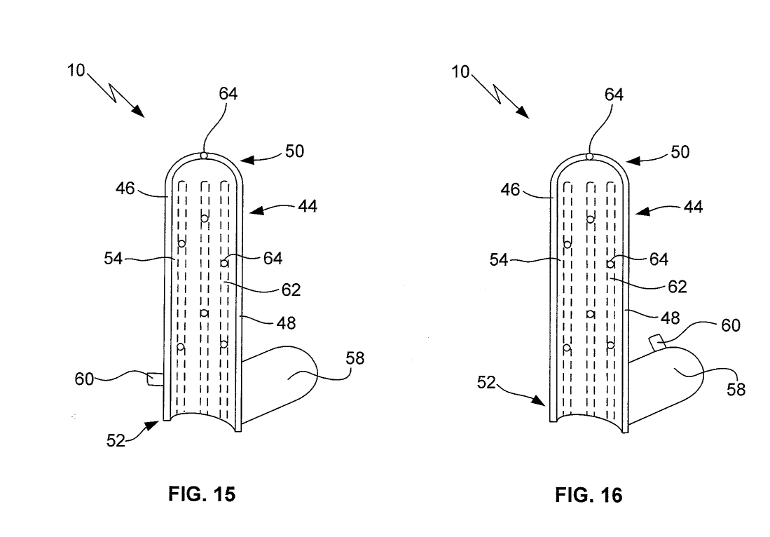

In an alternative embodiment, shown in FIGS. 15 and 16, guide 10 includes an injection port 60 hydraulically connected to one or more channels 62 disposed inside or along body member 44. Preferably, injection port 60 is configured to connect to or receive a syringe or like device for the injection of lubricant, water, saline solution or other fluid. Each of the channels 62 have one or more discharge openings 64 on inner side 54 for discharging fluid received into channels 62 from injection port 60. In a preferred embodiment, each channel 62 includes a plurality of discharge openings 64 along the longitudinal length of body member 44 and at or near its distal end 50 to discharge fluid, such as a lubricant, onto scope 14 and/or guide 10 to ease the movement of guide 10 along scope 14 or the scope 14 along guide 10. Preferably, the channels 62 are built into body member 44 so as to not be damaged during use or be damaging to the scope. As shown in FIG. 14, injection port 60 can be located at or near the proximal end 52 of body member 44. Alternatively, as shown in FIG. 16, injection port 60 can be placed on handle 58, which may provide some benefits as to the accessibility to injection port 60. If injection port 60 is located on handle 58, a handle channel, tube or like means, shown as 65 in FIG. 16, will have to be provided to hydraulically interconnect injection port 60 with channels 62 to discharge fluid out discharge openings 64. In the preferred embodiment, injection port 60 is adapted to connect to a syringe or other device for receiving fluid into channels 62. If inner side 54, or its liner 57, is coated with a hydrophilic lubricant, injection port 60 can be utilized to inject water or other fluid to enhance the lubricity of guide 10. Discharge openings 64 can all be the same size or be different sizes. Preferably, the size of discharge openings 64 is selected to allow the fluid injected into channels 62 to be distributed generally evenly along the inner side 54 of body member 44.

At one or more locations along the length of guide 10, particularly at its distal end 50 as shown in FIGS. 10 and 11, one or more ring members 66 can be incorporated into (i.e., integral with) or attached to body member 44 to substantially encircle the scope 14 during use for a colonoscopy. The ring members 66 are provided to ensure the guide 10 follows the scope 14 as it moves in the colon 12 and facilitates the pushing, pulling, and various lateral tilts that the guide 10 may impart to the scope 14. In a preferred embodiment, ring members 66 have a generally circular cross-section to encircle the scope 14 with an inner diameter only slightly larger than the outer diameter of the scope 14. Also in the preferred embodiment, ring member 66 has a longitudinally disposed slot 68, as shown in FIG. 11, along the length of ring member 66 to facilitate loading the guide 10 onto scope 14 when the scope 14 is already inside the colon 12. In use, the slot 68 is opened to receive the scope 14 inside ring member 66 in a side-loading configuration. The slot 68 is closed, with scope 14 inside ring member 66, by utilizing one or more commonly known closure mechanisms (not shown), such as adhesive tape that is wrapped around body member 44 at ring member 66. Another example of a closure mechanism is shown in U.S. Pat. No. 6,712,755 to Chang, which is incorporated herein by this reference. If ring member 66 is not provided with slot 68, then guide 10 must be preloaded onto the scope prior to its insertion into the colon 12. If desired, the ring members 66 can be coated with a hydrophilic substance or other lubricant to enhance the sliding of the scope 14 inside the ring members 66 and help prevent pinching of the mucosa of the colon 12. Additionally, the ring members 66 can be made out of a soft material in order to not scrape the scope 14 and be made collapsible to facilitate exchanging the scope 14 during the colonoscopy, as described below.

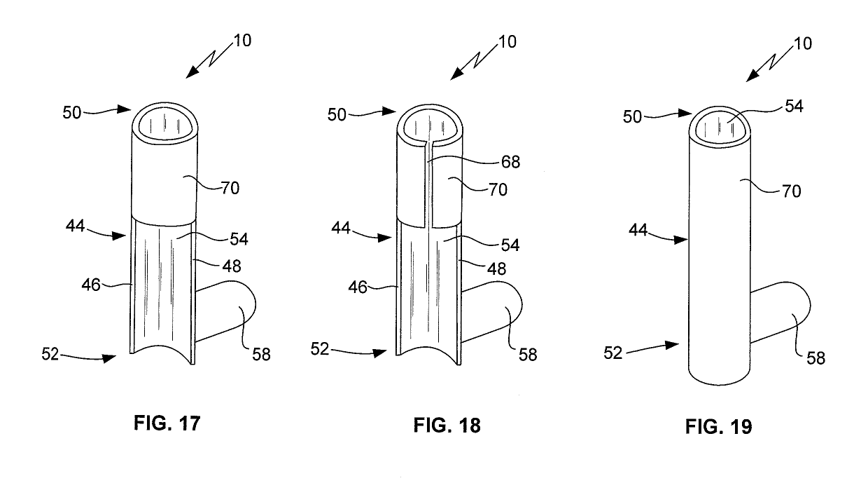

As an alternative to or in conjunction with the use of ring members 66, guide 10 can be provided with a short tube member 70, as shown in FIGS. 17 and 18, that is integral with or attached to body member 44 such that it resembles and functions similar to a short overtube/splint. In the preferred configuration of this embodiment, tube member 70 is an at least partially rigid tube that is placed at or near the distal end 50 of body member 44. In an alternative embodiment, shown in FIG. 19, tube member 70 can extend a substantial part or even all of the length of body member 44, essentially providing an overtube as guide 10. In use, the guide 10 having a non-slotted tube 70 (FIG. 17) is preloaded onto scope 14 by inserting scope 14 through tube member 70 and then sliding the guide 10 generally towards the proximal end (not shown) of scope 14 before beginning the colonoscopy. When the guide 10 is needed, the operator slides guide 10 distally to enter the rectum over the scope 14. If tube 70 is provided with a longitudinally disposed slot 68, such as shown in FIG. 18, then the need to preload guide 10 onto scope 14 is eliminated. As discussed above, a closure mechanism will be necessary to close slot 68 after loading scope 14 in tube 70.

Generally, the rigid component of body member 44 is approximately 5 to 25 cm. In another embodiment, particularly when utilized for the exchange of scopes 14, the length of the soft component of guide 10 can be longer, shorter or the same length as the rigid component, as long as it is of sufficient length to effectively protect the scope 14. In an alternative configuration, the soft component can be provided in segments. As will be readily apparent to those skilled in the art, guide 10 can have a variety of different configurations and width to accomplish the objectives set forth herein and accomplish the method of use set forth below. If desired, more than one of such configurations can be utilized during a colonoscopy procedure.

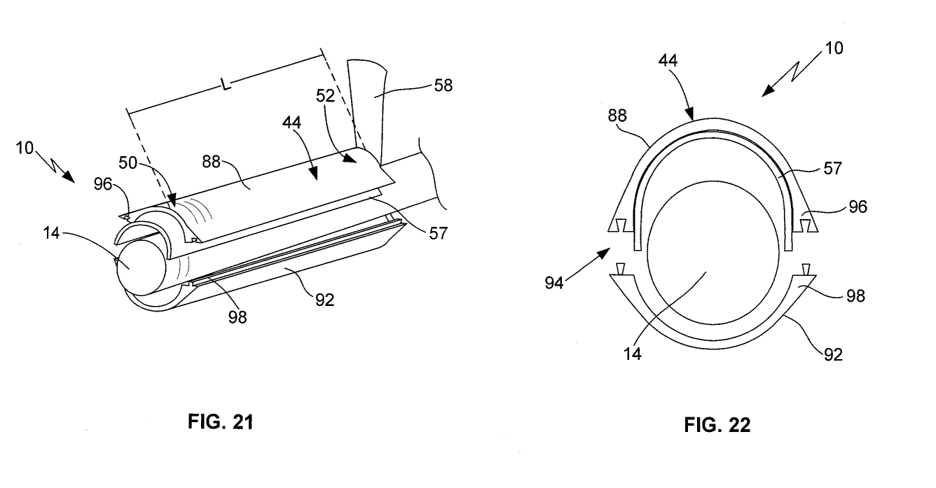

Another embodiment of the colonoscope guide 10 of the present invention is shown in FIGS. 21 and 22. In this embodiment, colonoscope guide 10 comprises an upper rigid member 88 (which in combination with lower rigid member 92, described below, defines body member 44) that is made out of a rigid material, such as stainless steel or a strong, rigid plastic so that it will be capable of tilting and maintaining the scope 14 in the rectum 20 in a generally oblique, leftward orientation. Although it is not necessary for the upper rigid member to be circular (i.e., surrounding scope 14), it should be wide enough to facilitate the desired tilting of scope 14 and to maintain the desired tilt as necessary. Attached at or near the proximal end 52 of upper rigid member 88 is handle 58, which is continuous with upper rigid member 88, being attached or integral thereto. As described in the embodiments above, handle 58 is configured to allow the user to tilt colonoscope guide 10 and to prevent complete entry of the upper rigid member 88 into the rectum 20. The rigid member 88 and handle 58 are cooperatively configured such that the insertion length (shown as L on FIGS. 21, 23 and 24), which is the length of the body member 44/rigid member 88 that is able to be inserted into the rectum 20 (e.g., the length of the body member 44/rigid member 88 between its distal end 50 and handle 58), is approximately 5 cm to 25 cm so as to be able to tilt the scope 14 while it is in the rectum 20 without the colonoscope guide 10 extending beyond the rectum 20. To prevent damage to the scope 14, at least the inner side 54 of upper rigid member 88 should be configured with liner 57, as described above and shown in FIGS. 21 and 22, that makes contact against the scope 14 without damaging it. In one preferred embodiment, the liner 57 is made out of silicone, rubber or the like and in the same shape as upper rigid member 88. As shown in FIG. 21, the upper rigid member 88 and liner 57 can have different lengths, with the liner 57 shown as being longer than the upper rigid member 88. However, to prevent damage to scope 14, the liner 57 should be at least the length of the upper rigid member 88.

To substantially encircle the scope 14, the embodiment includes a lower rigid member 92 that, in conjunction with upper rigid member 88, effectively forms body member 44 into a tube around scope 14. Although upper 88 and lower 92 rigid members can be integral, defining a full circle tube, it is preferred that the components be at least separable to allow the colonoscope guide 10 to be loaded onto the scope 14 only when it is needed to prevent or overcome looping or bending of the sigmoid 24. As known in the art, as set forth in prior art patents and devices, colonoscope guide 10 can be provided with a slotted tube with the slot to be closed and secured in the closed position with tape or the use of string, sutures or the like. For instance, the edges of the slot can be slanted, serrated or have a step-like configuration. Alternatively, the slot can remain as a gap that is then covered by a rigid piece of material. In the embodiment of FIGS. 21 and 22, the upper, rigid body member 44 and lower rigid member 92 are joined by a tube engaging mechanism 94 that is configured to allow the user to secure the scope 14 inside the colonoscope guide 10 when its use is needed. In this embodiment, the engaging mechanism 94 comprises an upper engagement member 96 on the body member 44 and a lower engagement member 98 on the lower member 92 that are shaped and configured to cooperatively engage each other so as to secure colonoscope guide 10 around the scope 14. In the embodiment of FIGS. 21 and 22, the engaging mechanism 94 is configured such that the upper 96 and lower 98 engagement members snap or lock together to securely form the tube around the scope 14. In another configuration, the upper engagement member 96 and lower engagement member 98 are generally U-shaped components that are in opposite facing directions to each other such that one is received in the other. In this manner, upper engagement member 96 and lower engagement member 98 can slidably engage each other. For example, the lower engagement member 98 can be configured to slide onto upper rigid member 88. If desired, the handle 58 can be located on the lower rigid member 92 instead of the rigid upper member 88. In this configuration, the operator of guide 10 would pull down on handle 58 to tilt the guide 10 toward the descending colon 26 while maintaining the liner 57 in contact against the scope 14.

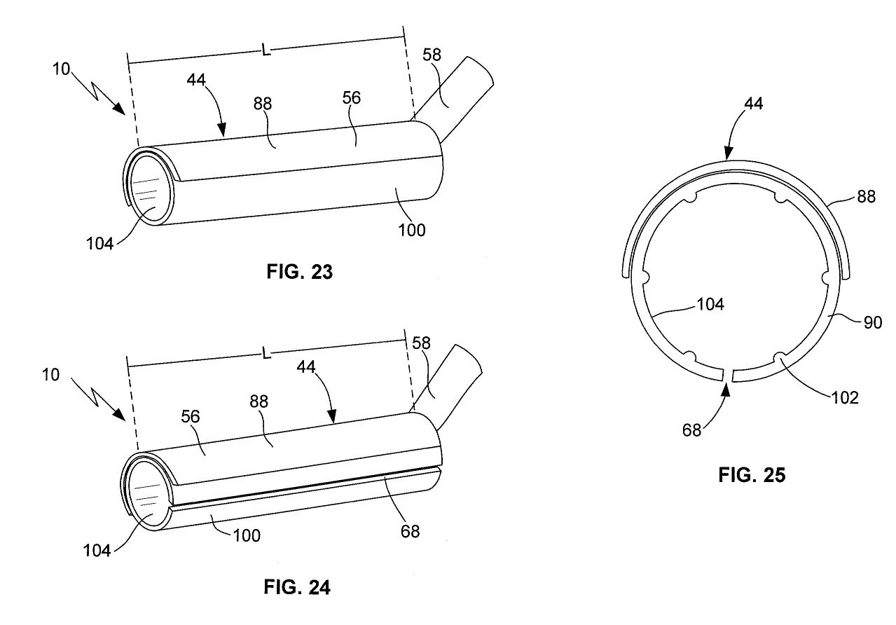

A preferred embodiment of guide 10 is shown in FIGS. 23 through 25. In this embodiment, body member 44 is just the upper rigid member 88, as shown in FIGS. 9 and 21-22, having a partial or semi-circular cross-section that is in abutting relation to an inner tubular member 100 that is made out of a softer material, such as silicone, rubber or the like, that is much less likely to damage the scope 14. In one embodiment, the upper rigid member 88/body member 44 encompasses approximately 30% to 50% of the circumference of the softer inner tubular member 100, which is a complete tube through which the scope 14 is positioned. Preferably, the soft inner tubular member 100 has a longitudinal slot 68, as shown in FIGS. 24 and 25, to allow side-loading of the scope 14. The guide 10 can be provided with a piece of adhesive tape that is removably attached to the outer surface of the guide 10. In a preferred configuration, the tape would be covered with a non-adhering paper. Once the scope 14 is loaded into the guide 10, the paper is peeled off and the adhesive tape is used to stick onto the outer surface of the guide 10, securely closing the slot 68. The handle 58 is continuous with the rigid upper member 88 to facilitate tilting of the scope 14 while inside the softer inner tubular member 100.

A cross-section of a preferred vector guide 10 is shown in FIG. 25. In this embodiment, the rigid outer member 88 encompasses approximately 50% of the circumference of the inner tubular member 100, which has a longitudinally disposed slot 68. To facilitate sliding movement between the guide 10 and scope 14, the guide 10 is provided with a plurality of small protrusions 102 along the inside wall 104 of inner tubular member 100. In one configuration, protrusions 102 are configured as “ribs” that extend longitudinally part or all of the length of the inner tubular member 100. Protrusions 102 serve to reduce friction and allow retention of some of the lubricant applied on the shaft of the endoscope 14, as it slides against the surface of the inside wall 104 of the tubular inner member 90.

Use of colonoscope guide 10 of the present invention can be initiated at either the beginning of the colonoscopy or upon reformation of a loop in the sigmoid 24 later in the procedure. During the initial introduction of the scope 14 through the sigmoid 24, with the lumen of the sigmoid 24 or descending colon 26 clearly seen straight ahead (which typically occurs when the tip 16 of the scope 14 is at the sigmoid-descending junction 38, looking ahead into the descending colon 26), the scope 14 is pulled back as much as possible. At this stage, the operator recognizes that further advancement of the scope 14 will require and depend upon formation of a substantial and/or painful sigmoid loop, despite the use of usual colonoscopic maneuvers. The solution is to use the guide 10 to tilt the scope 14 in the rectum 20 to the left and maintain the tilt, with adjustments in the angle, while pushing the scope 14 forward. The scope 14 will then advance without the painful sigmoid loop. In a preferred embodiment, this is accomplished by pulling the scope 14 as far back as possible without losing positioning inside the colon (to the extent possible), while keeping the lumen of the sigmoid 24 and/or descending colon 26 in view. The guide 10 is then introduced into the rectum 20 along the scope 14, and gently tilted leftward while lubricant or other fluid is applied. The lumen of sigmoid colon 24 or descending colon 26 is constantly kept in view by adjusting the bending section of the scope 14, while at the same time gently pushing the scope 14 to advance it into the lumen while lubricant is applied or injected through injection port 60. In doing so and by minimizing loop formation, the sigmoid 24 will be found to be very short, and the descending colon 26 and beyond will be reached quickly and with relatively little pain. If a loop is felt to form, the scope 14 can be pulled back again, and then the steps of tilting the guide 10 with lubricant application, tip adjustment and pushing are repeated as necessary or desirable.

A common situation arises when the tip 16 is in the sigmoid 24 or distal descending colon 26, looking straight ahead at the lumen, and yet pushing only causes a painful loop to form in the sigmoid 24. The guide 10 can be easily used to tilt the scope 14 in the rectum 20 leftward to be more in line with the descending colon 26, and then pushing the scope 14 will result in an efficient and relatively painless advancement of the scope 14 into the descending colon 26. In using this technique, there is no need to form an alpha loop, a maneuver to try to “soften” the angle of insertion of the scope 14 into the sigmoid 24 to lessen pain.

While usual colonoscopic maneuvers remain important, looping of the sigmoid 24 can be mitigated by the use of the guide 10 of the present invention. Thus, in addition to being an improved substitute for the prior art sigmoid splint/overtube device, the guide 10 can also help in the initial insertion of the scope 14 through the sigmoid 24.

In the scenario where the scope 14 is already advanced to the descending colon or beyond when a loop in the sigmoid 24 forms, the scope 14 is pulled back to undo the sigmoid loop, before further advancement. As known to those skilled in the art, at certain point or points during the colonoscopy when the scope 14 in the descending 26, transverse 30 or ascending 32 colon, pushing causes the scope 14 to “buckle” in the sigmoid 24, and the tip 16 fails to advance or even moves backward. This feeling is very familiar to all colonoscopists or other operators. It can be quite a struggle, with the usual colonoscopic maneuvers being ineffective. The sigmoid loop can reform when pushing the scope 14 against increased frictional resistance. The hepatic flexure is especially problematic as it is suspected that at that point, much of the lubricant at the distal aspect of the scope 14 has been deleted by contact with the colon wall. When such resistance occurs, pushing the scope 14 causes the rectum 20, slightly tilted by straightening the scope 14, to go back to its craniocaudal axis. Further pushing of the scope 14 thus sets off the same chain of events. With the scope 14 pulled back to straighten again, the scope 14 in the rectum 20 can again be readily tilted leftward by using the guide 10. With the scope 14 held in this oblique axis, the force of pushing will now go directly in line with the descending colon 26, and the forward force for scope 14 advancement is transmitted much more effectively to its tip 16, as shown in FIG. 8.

Thus, as set forth above, the vector guide 10 can be used multiple times throughout a colonoscopy procedure. The guide 10 can be used on initial insertion and then again and again subsequently. Along with the usual colonoscopic maneuvers of the scope, the guide and the scope should be repeatedly lubricated, tilted, advanced and withdrawn as needed. As the procedure becomes much quicker, easier, and more predictable, cecal intubation rate also increases, while pain, and its associated consequences, are lessened.

In summary, the craniocaudal axis of the pushing force causes sigmoid looping. By tilting the axis of scope 14 during insertion, looping can be minimized through the sigmoid 24. Later into the procedure, sigmoid looping can also be avoided using this same method, as the scope 14 in the rectum 20 is kept in line with the descending colon 26. As stated above, from the configuration of FIG. 3, the prior art colonoscopy usually goes to FIG. 4, a very painful move for the patient. Using the guide 10 and method of the present invention, however, the procedure would go to FIG. 5 instead, which is far more desirable for the patient than FIG. 4. Similarly, from FIG. 6 the prior art colonoscopy may go to FIG. 7, whereas utilizing the guide 10 and method of the present invention, the procedure would go to FIG. 8, also a far more desirable configuration.

During further advancement of scope 14, a deep transverse colon loop is sometimes encountered. If the sigmoid looping is prevented, the deep transverse colon loop is much less of a problem. Sometimes, a longer guide 10, such as one approximately 40 to 50 cm (similar in length in to an overtube or even longer) which can be inserted to the descending colon 26 or even the transverse colon 30, can be helpful. The shorter guide 10, if used, can be removed, and exchanged for a longer guide 10. The longer length body member 44 is made of a short rigid component (i.e., rigid upper member 88) that is 5 cm to 25 cm in length, and a long soft component (i.e., soft inner layer 90).

If scope 14 needs to be removed, such as when retrieving a large polyp after polypectomy, a longer guide 10 may also be helpful. The scope 14 is removed while the longer guide 10 is kept in place in the descending colon 26.

When the scope 14, or another scope, is re-introduced, it can be introduced easily inside guide 10, back into the descending colon 26, without having to work its way through the turns of the sigmoid 24 again. In this situation, a long guide 10 with a ring member 66 at its distal end 50 can be used. The ring member 66 can be made of collapsible material, such that on reinsertion of the scope 14, the scope 14 does not have to be reinserted through the ring member 66. Once the scope 14 is in the descending colon 26, the guide 10 can be withdrawn and removed with the ring member 66 collapsed. The exchange of scopes maneuver can also be carried out easily if difficulty is encountered upstream and a hydrophilic sleeve (i.e., a sleeve member coated with a hydrophilic substance that is placed over the scope 14) is deemed desirable. The scope 14 is removed in this manner, loaded with the hydrophilic sleeve, and then reintroduced.

Occasionally, a prior art sigmoid splint/overtube is inserted over the scope 14. If it is successful, it can be very helpful in the advancement of the scope 14. However, sometimes insertion of the overtube meets resistance at the recto-sigmoid junction because there is still a bend there despite pulling the scope 14 back. At that point, the simple solution, offered with the guide 10 and method of the present invention, is to introduce the guide 10 to tilt the scope 14 in the rectum 20 to the left, and maintaining that axis as the scope 14 is pushed forward. The scope 14 from the anus 22 to descending colon 26 is kept in a relatively straight line. Buckling is avoided, and looping of the sigmoid 24 is also avoided, as shown in FIGS. 5, 6 and 8, while the scope 14 moves forward efficiently. The guide 10 and method of the present invention thus can serve the same purpose as a sigmoid splint/overtube, except it is much more easily loaded onto the scope 14, it is much easier to use (i.e., not hampered by a bent recto-sigmoid junction), and it is safer. While the purpose is similar, namely maintaining straightness from the anus 22 to the descending colon 26, the prior art overtube does not provide for or offer active tilting due to its necessary flexibility. In addition, prior art overtubes cannot be utilized to facilitate the initial scope 14 insertion through the sigmoid 24. Further, although the guide 10 can include ring member 66 or short tubular member 70, a totally tubular configuration is not required, as it is for the standard overtube. A complete tubular member, such as inner tubular member 100 (FIGS. 23 and 24) may be advantageous, however, as it tends to maintain the scope 14 in a substantially straight line, three-dimensionally. As described above, the rectum 20 curves posteriorly along the sacrum, which can cause the endoscope 14, upon insertion, to follow this same curvature.

In another method of use of the guide 10 of the present invention that is configured with a plurality of ring members 66 or one or more tube members 70, is to first insert at least a portion of body member 44 of guide 10 into the rectum and then insert the scope 14 into engagement with ring members 66 or inside tube 70. The scope 14 and the body member 44 of guide 10 are then advanced together while the operator tilts the guide 10 in the general direction of the descending colon 26 and manipulates the scope 14 to ease the passage of insertion tube 18 through the colon 12.

As the guide 10 is tilted leftward, it exerts pressure on the endoscope 14. Lubricity is required for movement of the endoscope 14, against this pressure. One solution is to provide little “bumps” or protrusions 102 (shown in FIG. 25) on the inside wall 104 of the soft inner tubular member 100. These protrusions 102 may be extended longitudinally or in other configurations to form small, longitudinal ridges. These small protrusions 102 are elevated on an otherwise smooth surface of inside wall 104 help to reduce the surface area of contact with the endoscope 14, and also help retain some of the lubricant applied onto the surface of the endoscope 14.

It is anticipated that guide 10 of the present invention can also be utilized for medical procedures other than a colonoscopy that require the insertion of scope 14 or like medical device into the human body and where it is necessary or desirable to tilt the scope 14 so as to direct it to its target location. For instance, guide 10 is likely to be useful in a natural orifice transluminal endoscopic surgery (commonly referred to as NOTES), such as the splenectomy shown in FIG. 20. In addition to the colon, this figure shows the stomach 80, duodenum 82, spleen 84 and greater omentum 86. As shown in FIG. 20, scope 14 is inserted into the rectum 20 and then directed through an incision generally located near the recto-sigmoid junction 36 into the peritoneal cavity. Guide 10, preferably in the form of a rigid overtube, is introduced into the rectum 20 over the scope 14. Using guide 10, the operator tilts the axis of the scope 14 toward spleen 82 to perform the splenectomy or tilt to the right to perform a cholecystectomy.

As set forth above, the scope 14 has to move in and out, often with torquing or rotation of the scope shaft, while a considerable force is exerted upon the surface of the scope 14 in order to tilt the scope 14 leftward. The friction thus generated is especially marked at the distal end 50 of the guide 10. If the distal end 40 of the guide 10 is composed of hard material, such as metal or a hard plastic, it will tend to scrape the surface of the scope 14 like a blunt knife. This scraping of the scope 14 is especially pronounced when the scope 14 is pulled against the tilted guide 10. The edge at the distal end 50 of the guide 10 will act like a blunt knife scraping against the surface of the scope 14 as the scope 14 moves against the edge. This scraping impedes movement of the scope 14. Even worse, scraping and abrading against the edge at the distal end 50 of the guide 10 can cause severe damage to the surface of the scope 14. This damage appears as a scrape or cut on the surface of the scope 14, and causes leakage from the scope 14 and blurring of the image of the colon 12. A single repair of this damage to the scope 14 often amounts to thousands of dollars. Lubricants applied on a guide 10 made of hard material can lessen these effects, but it is not likely to prevent damage to the scope 14. Therefore, a soft liner 57 or use of soft inner tubular member 100 is beneficial to use of the guide 10. In a preferred embodiment, the soft liner 57 or soft inner tubular member 100 that cushions the otherwise hard surface contact from the inner side 54 of the guide 10, especially at its distal end 50, is incorporated into the construction of the guide 10.

Hence, it is necessary to construct a guide 10 with unique features not present in the existing anoscopes or sigmoidoscopes to carry out this leftward tilting function effectively, smoothly, and without damage to the scope 14. The following features are disclosed and are, preferably, incorporated into the guide 10 of the present invention:

1) The guide 10 has an elongated body member 44 to carry out the tilting action in the rectum 20;

2) The guide 10 has to be sufficiently rigid longitudinally along its length to be able to exert the needed leftward force upon the scope 14 in the rectum 20 and the cross-sectional width of the rigid outer side 56 has to be sufficient to carry out this tilting action;

3) The guide 10 has a handle 58 at its proximal end 52 to prevent its complete entry into the rectum 20 and to facilitate the tilting action;

4) The inner side 54 of the guide 10, at least at or towards its distal end 50, has to have a relatively soft material, either liner 57 of the soft inner tubular member 100, but they do not have to be the same length as the outer, rigid side 56 of body member 44 or upper rigid member 88;

5) Lubricity of the surface of the inner side 54 of the guide 10, preferably provided by a hydrophilic coating, is preferred to allow smooth movement of the scope 14 and/or protrusions 102, such as ridges or bumps, can be incorporated onto the surface of the inner side 54 to reduce the contact surface and, hence, friction against the scope 14;

6) The use of a longitudinal slot 68, which opens up for side-loading of the scope 14 and then closes to house the scope 14 and allow for atraumatic insertion through the anus 22, in the body member 44 is highly desirable;

7) The guide 10 is preferably tubular shaped in order to avoid traumatizing the anus 22 of the patient; and

8) The outer side 56 of the distal end 50 of the guide 10 preferably is tapered to allow easy, atraumatic insertion through the anus 22.

Those skilled in the art will appreciate that the advantages of a guide 10 that facilitates leftward tilting of the scope 14 within the rectum 20 are: (1) by working within the rectum 20 alone, the guide 10 can aid advancement of the scope 14 through the sigmoid 24 and descending colons 26; (2) the device does not have to cross the recto-sigmoid junction 36, thereby making perforations extremely unlikely; (3) the rigidity and short length of the guide 10 make a longitudinal slot 68 more feasible, such that side-loading can be accomplished, which is highly desirable because the scope 14 does not have to be removed from the colon 12 in order to load the guide 10; and (4) the short length takes up much less length of the scope 14, so that handling of the scope 14 is much easier, compared to the prior art longer, bridging sigmoid splints.

As set forth above and shown in FIGS. 23 through 25, in a preferred embodiment the guide 10 comprises a soft inner tubular member 100 that is tubular shaped and placed against scope 14. Preferably, the soft inner tubular member 100 is constructed of a material that substantially eliminates or at least minimizes abrasion of the scope 14 when the scope 14 slides against the guide 10, such as silicone, rubber or the like. The body member 44, comprising the upper rigid member 88, can have a tapered distal end 50 to ease introduction into the rectum 20 through the anus 22. A hydrophilic coating can be placed onto the surface of the inside wall 104 of the soft inner tubular member 100 for enhanced lubricity. A longitudinal slot 68, shown in FIGS. 24 and 25, can be provided to allow side-loading of the device 10 onto the scope 14.

An elongated outer body member 44, which is constructed of a material that is sufficiently rigid to be able to exert a leftward force upon the scope 14 in the rectum 20, is adhered or otherwise placed in abutting relation to the soft inner tubular member 100. As shown in FIGS. 23 through 25, this outer body member 44 can be configured to be roughly semi-circular in cross-section. A handle 58 constructed of the same material or another material of similar rigidity is attached to or integral with body member 44 at or near the proximal end 52 thereof. The handle 58 can be continuous with the body member 44, but it should extend outward at an angle to the body member 44 in order to facilitate performance of the tilting action.

In one embodiment, shown in FIG. 23, longitudinal slot 68 is absent from inner tubular member 100, which will require scope 14 to be end-loaded and will not permit side-loading. In this configuration, body member 44 can have a generally semi-circular cross-section, as shown, or a fully circular cross-section.

In another embodiment, shown in FIGS. 21 and 22, body member 44 can comprise a rigid upper member 88 and a rigid lower member 92 that are provided with a soft liner 57 at the inner side wall of the upper member 88 that abuts the scope 14 to reduce the likelihood of damage to the scope 14 caused by abrasion or the like.

In another embodiment, the body member 44 is tubular shaped, such as shown in FIG. 19, and is constructed of a single material that is selected so as to be sufficiently soft and non-abrasive, such as silicone or the like, so as to avoid scraping or otherwise damaging the scope 14 without the need for a liner 57 or inner tubular member 100. The body member 44 can have a relatively thick wall, particularly on the side toward handle 58, to provide the necessary stiffness to be able to tilt the scope 14 in the rectum 20. By using a thick wall of the soft material, sufficient longitudinal rigidity of the tubular body member 44 can be achieved to be able to tilt the scope 14 in the rectum 20 without the need for any additional stiff layers (e.g., rigid upper member 88).

While there are shown and described herein certain specific alternative forms of the invention, it will be readily apparent to those skilled in the art that the invention is not so limited, but is susceptible to various modifications and rearrangements in design and materials without departing from the spirit and scope of the invention. In particular, while the above description contains many specifics, these should not be construed as limitations on the scope of the invention, but rather as an exemplification of one or more preferred embodiments thereof. For instance, the references to scope 14 above include any type of endoscope that is or may be used to perform a colonoscopy or like medical procedure. Further, it should be noted that the present invention is subject to modification with regard to assembly, materials, size, shape and use. Some of the components described herein can be made integral with each other to reduce the number of separate components. For instance, the handle 58, injection port 60, ring members 66 and/or tube 70 can be made integral with body member 44.

What is claimed is:

1. A colonoscope guide for use with an endoscope in a colonoscopy procedure, said colonoscope guide comprising:
an elongated body member having a proximal end, a distal end and an inner side, said body member sized and configured to be received in a rectum through an anus, said body member sufficiently rigid to tilt the endoscope while said body member and the endoscope are in the rectum, said body member having an insertion length selected to prevent said body member from extending beyond the rectum when said body member is in said rectum; and
a handle at or near said proximal end of said body member and in continuity with said body member, said handle sized and configured to facilitate tilting of said body member from outside the anus while said body member is in the rectum and to prevent full entry of said guide into the rectum.

2. The guide of claim 1, wherein said body member is tubular along at least a portion of its length.

3. The guide of claim 2, wherein said body member has a longitudinal slot configured to removably receive the endoscope inside said tubular body member.

4. The guide of claim 1, wherein said body member is non-tubular and has a generally curved cross-section.

5. The guide of claim 4 further comprising a liner, said liner being generally soft and positioned against said inner side of said body member so as to contact the endoscope without damage to the endoscope.

6. The guide of claim 4, wherein said body member is a rigid upper member having a generally semi-circular cross-section.

7. The guide of claim 6 further comprising an inner tubular member positioned against said inner side of said rigid upper member, said inner tubular member being generally flexible and configured to receive the endoscope therein, said rigid upper member configured to tilt the endoscope while the endoscope is inside said inner tubular member.

8. The guide of claim 7, wherein said inner tubular member has a plurality of protrusions on an inner wall thereof configured to increase lubricity and retention of lubricant.

9. The guide of claim 8, wherein said inner tubular member has a longitudinal slot configured to removably receive the endoscope inside said inner tubular member through said longitudinal slot.

10. The guide of claim 7, wherein said inner tubular member has a longitudinal slot configured to removably receive the endoscope inside said inner tubular member through said longitudinal slot.

11. The guide of claim 10, wherein said inner tubular member is sufficiently flexible to receive the endoscope through said longitudinal slot.

12. The guide of claim 1, wherein said body member comprises a rigid upper layer, a rigid lower layer and means for engaging said upper layer to said lower layer to substantially encircle the endoscope.

13. The guide of claim 12 further comprising a liner, said liner being generally soft and positioned against said rigid upper layer so as to contact the endoscope without damage to the endoscope.

14. The guide of claim 1 further comprising an injection port at said proximal end of said body member and/or said handle, one or more discharge openings on said inner side of said body member configured to discharge fluid onto said inner side of said body member and/or the endoscope, and one or more channels disposed in said body member hydraulically interconnecting said port and said one or more discharge openings.

15. The guide of claim 1 further comprising one or more ring members attached to or integral with said body member, said one or more ring members sized and configured to receive the endoscope.

16. The guide of claim 15, wherein each of said one or more ring members has a longitudinal slot sized and configured to receive the endoscope through said slot.

17. A colonoscope guide for use with an endoscope in a colonoscopy procedure, said colonoscope guide comprising:
an elongated rigid body member having a proximal end, a distal end and an inner side, said body member sized and configured to be received in a rectum through an anus, said body member sufficiently rigid to tilt the endoscope while said body member and the endoscope are in the rectum, said body member having an insertion length selected to prevent said body member from extending beyond the rectum when said body member is in said rectum;
a handle at or near said proximal end of said body member and in continuity with said body member, said handle sized and configured to facilitate tilting of said body member from outside the anus while said body member is in the rectum and to prevent full entry of said guide into the rectum; and
an inner tubular member positioned against said inner side of said body member, said inner tubular member being generally flexible and configured to receive the endoscope therein, said body member configured to tilt the endoscope while the endoscope is inside said inner tubular member.

18. The guide of claim 17, wherein said inner tubular member has a longitudinal slot configured to removably receive the endoscope inside said inner tubular member through said longitudinal slot.

19. The guide of claim 18, wherein said inner tubular member is sufficiently flexible to receive the endoscope through said longitudinal slot.

20. The guide of claim 17, wherein said inner tubular member has a plurality of protrusions on an inner wall thereof configured to increase lubricity and retention of lubricant.
